(19) United States
(12) Patent Application Publication
(10) Pub. No.: US 2010/0174315 A1
(43) Pub. Date: Jul. 8, 2010

(54) DEVICE FOR SPINAL FUSION

(76) Inventors: Daniel Scodary, Bridgeton, MO (US); Paul M. Sand, Redwood City, CA (US)

Correspondence Address: Polster, Lieder, Woodruff & Lucchesi, L.C., 12412 Powerscourt Dr. Suite 200, St. Louis, MO, 63131-3615, US

(21) Appl. No.: 12/639,699

(22) Filed: Dec. 16, 2009

Related U.S. Application Data
(60) Provisional application No. 61/138,022, filed on Dec. 16, 2008.

(51) Int. Cl. A61B 17/70

(52) U.S. Cl. 606/248; 606/264

(57) ABSTRACT

A device for covering and protecting the spinal cord of a patient after some or all of the spinous process or lamina has been removed. The device includes a main body adjustably attached to at least two rod attachment flanges such that each rod attachment flange may be adjusted closer and further from the main body. The rod attachment flanges are each attached to a pedicle rod having at least four pedicle screws attached thereto. The pedicle attachment screws are adapted for attachment to at least one vertebrae of the patient.

### CROSS-REFERENCE TO RELATED APPLICATIONS

The present application claims priority to U.S. Provisional Patent Application Ser. No. 61/138,022 filed Dec. 16, 2008, the contents of which are incorporated herein by reference.

### FIELD OF THE INVENTION

This invention relates generally to the field of spinal fusion and more specifically to instrumentation for spinal fusion and maintenance of the musculature around the spine.

### BACKGROUND OF THE INVENTION

Spinal fusion is a surgical technique used to combine two or more vertebrae. Supplementary bone tissue (either autograft or allograft) is used in conjunction with the body's natural osteoblastic processes. This procedure is normally used to eliminate the pain caused by abnormal motion of the vertebrae by immobilizing the vertebrae themselves.

Spinal fusion is performed most commonly in the lumbar region of the spine, but it is also used to treat cervical and thoracic problems. Patients requiring spinal fusion have either neurological deficits or severe pain which has not responded to conservative treatment.

There are two main types of lumbar spinal fusion, which may be used in conjunction with each other:

Posterolateral fusion places the bone graft between the transverse processes in the back of the spine. These vertebrae are then fixed in place with screws and/or wire through the pedicles of each vertebra attaching to a metal rod on each side of the vertebrae.

Interbody fusion places the bone graft between the vertebra in the area usually occupied by the intervertebral disc. In preparation for the spinal fusion, the nucleus pulposus is removed entirely. A device may be placed between the vertebra to maintain spine alignment and disc height. The intervertebral device may be made from either plastic or titanium. The fusion then occurs between the endplates of the vertebrae. Using both types of fusion is known as 360-degree fusion. Fusion rates are higher with interbody fusion.

In most cases, the fusion is augmented by a process called fixation, meaning the placement of metallic screws (pedicle screws often made from titanium), rods or plates, or cages to stabilize the vertebra to facilitate bone fusion. The fusion process typically takes 6-12 months after surgery.

Spinal instrumentation and fusion are not new surgical concepts. Although the first spinal fusion was performed almost 90 years ago, Dr. Paul Harrington developed spinal instrumentation in the late 1950's. It is estimated that roughly 220,000 Americans undergo spinal fusion each year.

### BRIEF SUMMARY OF THE INVENTION

The present invention comprises a device for covering and protecting the spinal cord of a patient after some or all of the spinous process or lamina has been removed. The device comprises a main body adjustably attached to at least two rod attachment flanges such that each rod attachment flange may be adjusted closer and further from the main body. The rod attachment flanges are each attached to a pedicle rod having at least four pedicle screws attached thereto. The pedicle attachment screws are adapted for attachment to at least one vertebrae of the patient.

### BRIEF DESCRIPTION OF THE SEVERAL VIEWS OF THE DRAWINGS

FIG. 1 is an end view of an improved device for spinal fusion according to a first embodiment of the present invention;

FIG. 2 is a side view of an improved device for spinal fusion according to a first embodiment of the present invention;

FIG. 3 is a perspective view of an improved device for spinal fusion according to a first embodiment of the present invention;

FIG. 4 is an end view of an improved device for spinal fusion according to a second embodiment of the present invention;

FIG. 5 is a side view of an improved device for spinal fusion according to a second embodiment of the present invention;

FIG. 6 is a perspective view of an improved device for spinal fusion according to a second embodiment of the present invention;

FIG. 7 is a perspective view of an improved device for spinal fusion according to a second embodiment of the present invention;

FIG. 8 is a close up view of an improved device for spinal fusion according to a second embodiment of the present invention;

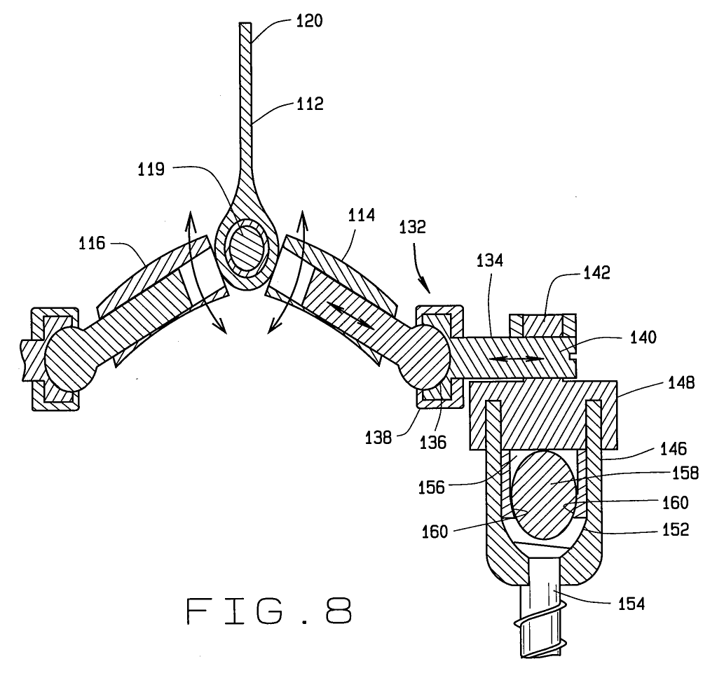

FIG. 9 is an exploded view of an improved device for spinal fusion according to a second embodiment of the present invention;

FIG. 10 is a close up view of an improved device for spinal fusion according to a third embodiment of the present invention;

FIG. 11 is a perspective view of an improved device for spinal fusion according to a third embodiment of the present invention;

FIG. 12 is a side view of an improved device for spinal fusion according to a third embodiment of the present invention;

FIG. 13 is an end view of an improved device for spinal fusion according to a third embodiment of the present invention; and

FIG. 14 is an exploded view of an improved device for spinal fusion according to an embodiment of the present invention.

### DESCRIPTION OF THE PREFERRED EMBODIMENT

While this invention is susceptible of embodiment in many different forms, there is shown in the drawings and will herein be described in detail preferred embodiments of the invention with the understanding that the present disclosure is to be considered as an exemplification of the principles of the invention and is not intended to limit the broad aspect of the invention to the embodiments illustrated.

The present invention comprises an improved instrumentation for spinal fusion that is fully adjustable and provides a sloped roof and muscle attachments for maintaining musculature in proper position and away from the spinal cord after surgery. The device includes an anatomically shaped arch that covers and protects the spinal cord after some or all of the spinous process and lamina have been removed.

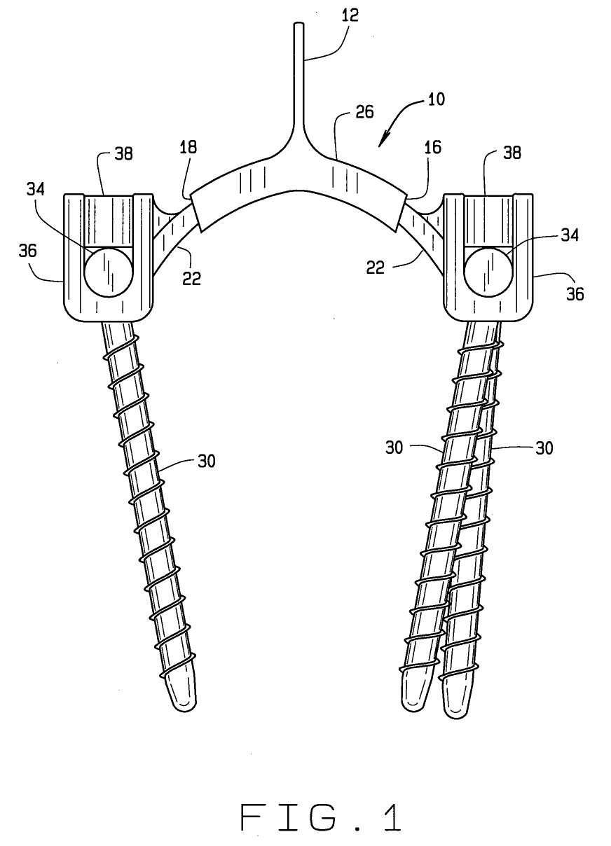

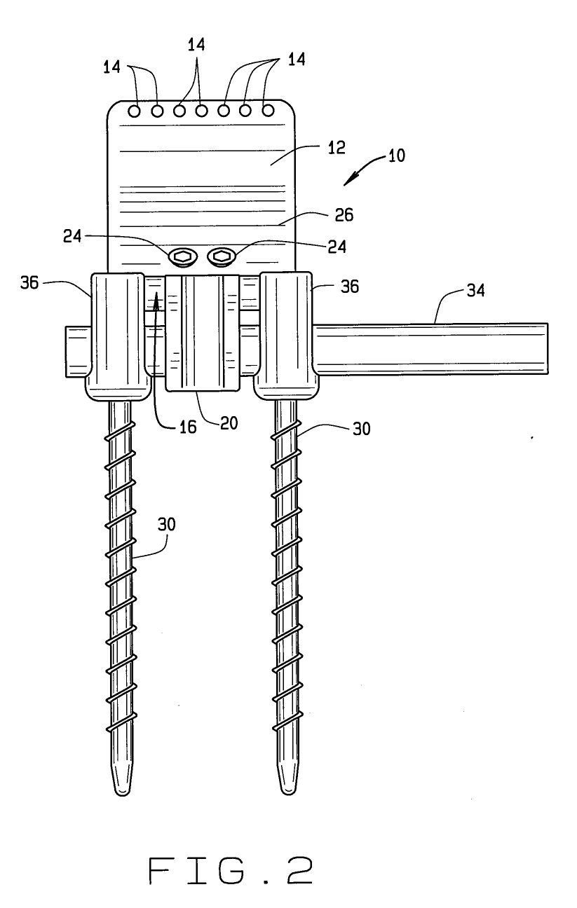

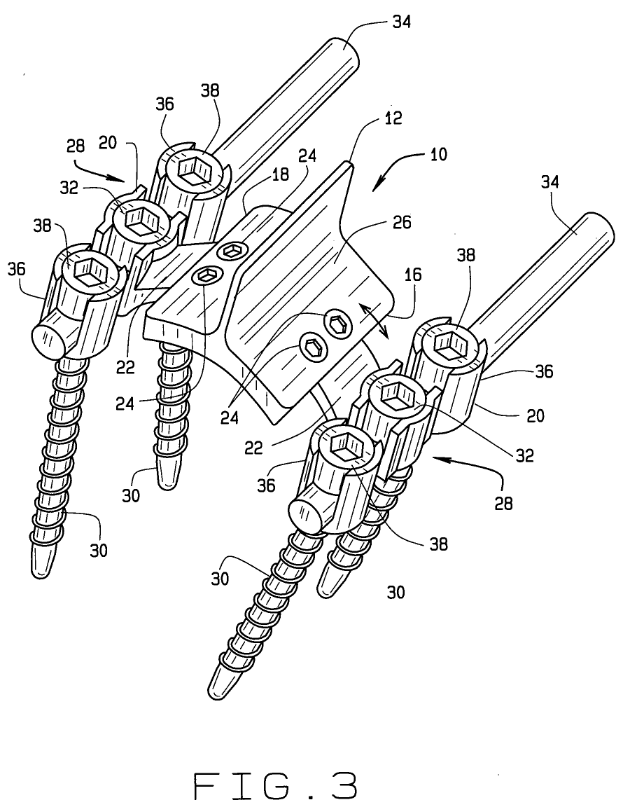

In that regard and referring to FIGS. 1-3, an embodiment of the present invention is shown. The embodiment includes a main body 10 which is arch-shaped and includes an extension portion 12 extending outwardly therefrom. The extension portion defines a plurality of bores 14 which may be used for suture attachment. Rod attachment flanges 20 are adjustably attached to opposite sides 16 and 18 of the main body 10. An extension 22 of each of the rod attachment flanges 22 is received in a slot formed within the sides 16 and 18 of the main body. Set screws 24 threaded into the top 26 of the main body 10 fix the extensions 22 in a desired location.

Attached to each extension 22 of the rod attachment flanges 20 is a pedicle rod attachment portion 28. In a preferred embodiment, a pedicle rod 34 is maintained in position by the pedicle rod attachment portion 28 by use of set screws 32 that thread into the pedicle rod attachment portion 28. The set screws 32 when tightened exert a clamping action on the pedicle rod 34 to maintain the pedicle rod 34 and the pedicle rod attachment portion 28 in a fixed relationship. When loosened, the set screws 32 release the clamping force and allow the pedicle rod 34 to move to be moved axially within the pedicle rod attachment portion 28 for adjustment of the location of the main body 10 with respect to pedicle screws 30 (described further below).

Further attached to each pedicle rod 34 are a pair of pedicle screw attachment flanges 36. The pedicle screw attachment flanges 36 attach pedicle screws to the main body 10 by utilizing set screws 38 to both clamp onto the pedicle rod 34 and maintain each pedicle screw 30 in a fixed relationship to the main body 10 when the set screw 38 is tightened. When the set screw 38 is loosened the pedicle screw 30 may be moved to adjust the angle of the pedicle screw 30 to the pedicle screw attachment flange 36. Additionally, when the set screw 38 is loosened, the pedicle screw attachment flange 36 and pedicle screw 30 combination may be moved axially along the pedicle rod 34 to adjust the location of the pedicle screw 30 with respect to the other screws 30 and with respect to the main body 10. The main body most preferably comprises an inert material or comprises a coating suitable for placement within the human body, and more preferably comprises a coating of a flexible, inert, medical-grade silicone elastomer. One example of a suitable elastomer is available under the trademark SILASTIC available from the Dow Corning Company of Midland, Mich. The coating also preferably comprises a growth factor to promote both bone and musculature regeneration and healing.

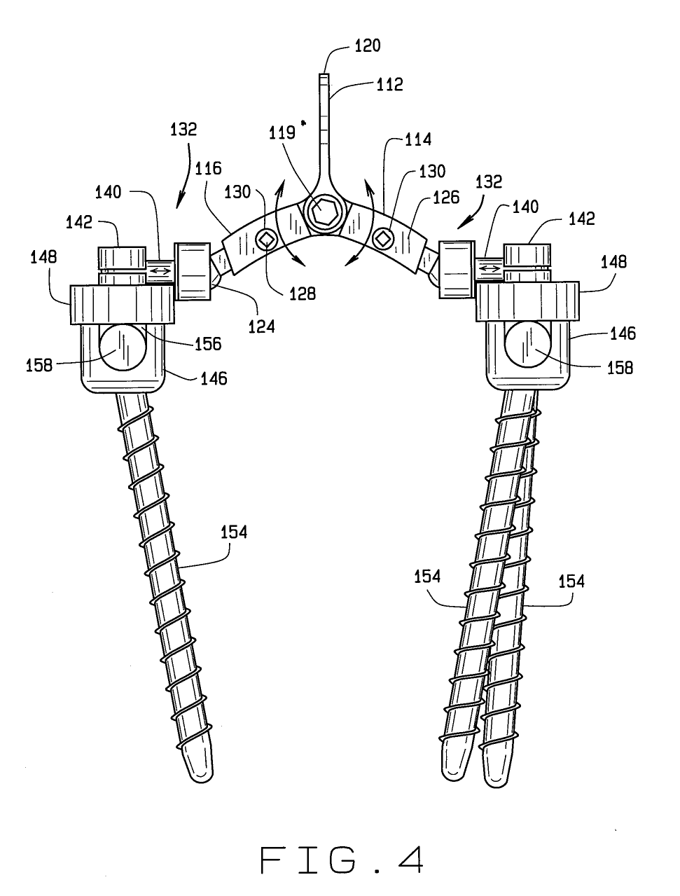

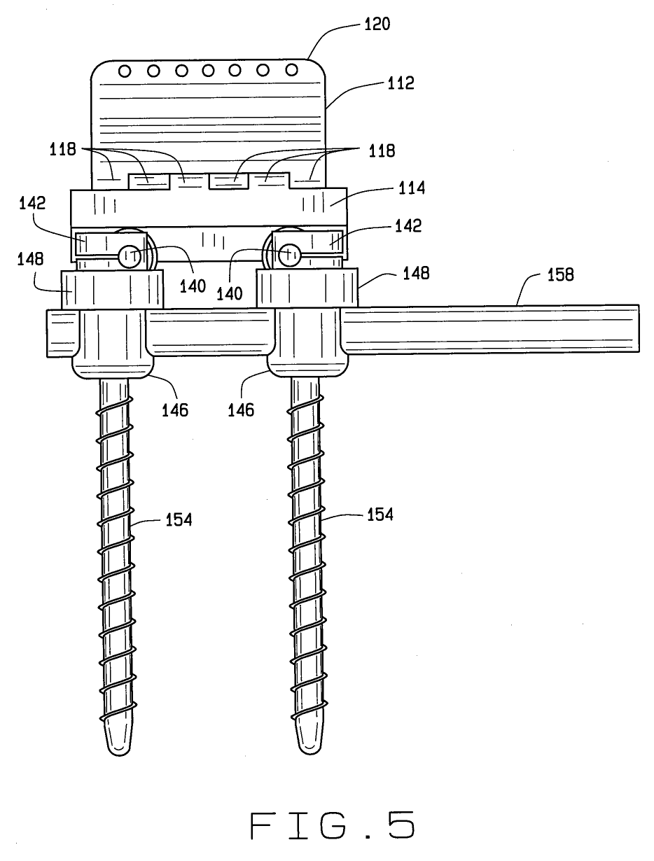

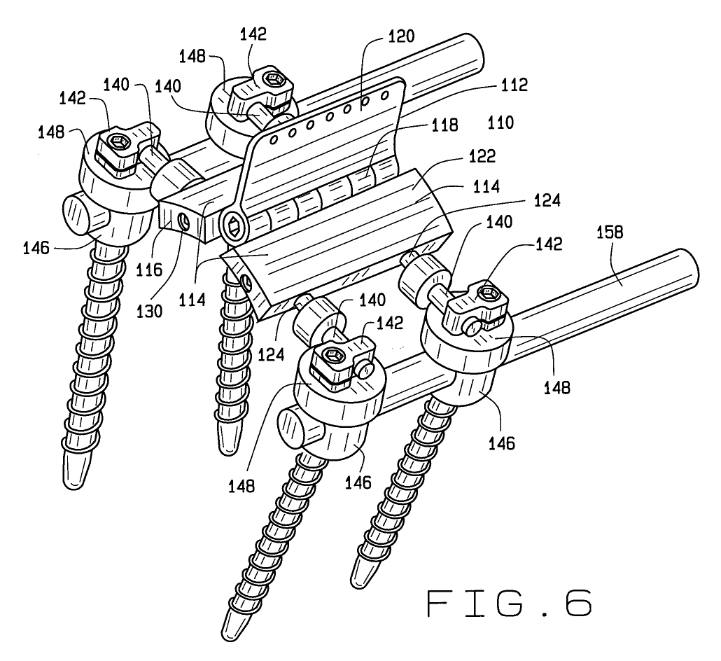

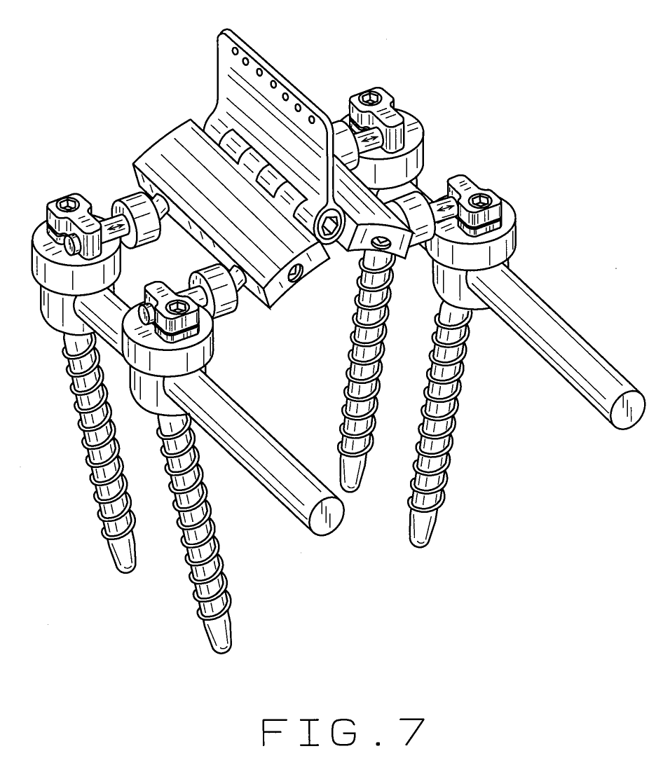

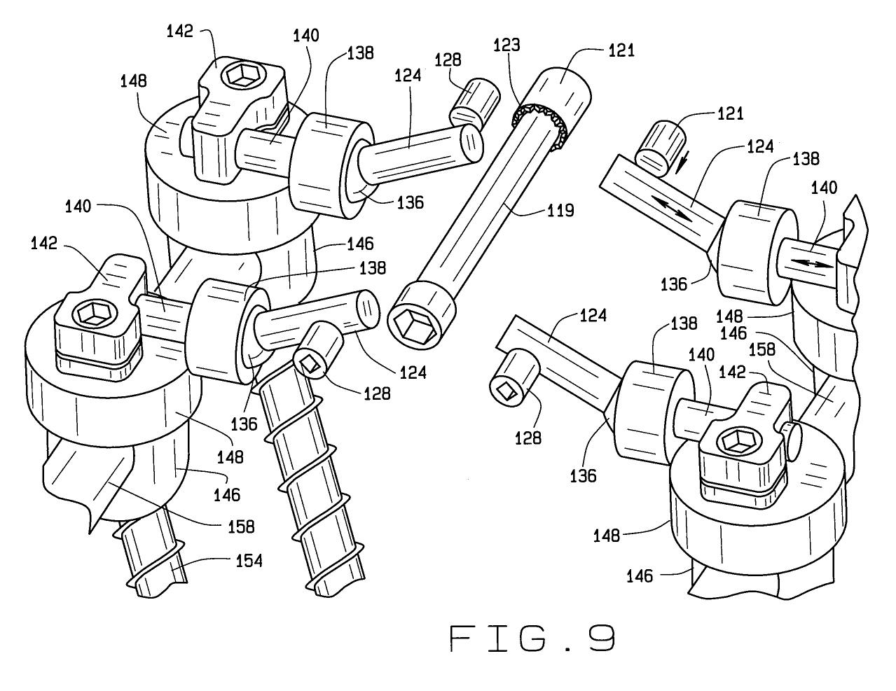

Referring to FIGS. 4-7, there is provided another embodiment of the present invention. The second embodiment comprises a modular main body 110 comprising hinged members 112, 114 and 116. The hinged members are attached by interdigitated hinge legs 118 and a hinge pin comprising a screw 119 that threads into a nut or into an end hinge leg 118. Most preferably, the nut 121 comprises a tapered nut (see FIG. 9) within a tapered bore of a hinge leg 118. The nut may further comprise projections 123 that prevent the screw 119 and nut 121 from inadvertently loosening. A first hinge member 112 comprises an extension portion 120, and second and third hinge members 114 and 116 comprise a main body 122. When the screw 119 is tightened, the interdigitated legs 118 are forced into a clamped relationship preventing relative rotation of the hinged members 112, 114 and 116 with respect to one another. When the screw 119 is loosened, the hinged members 112, 114 and 116 are allowed to rotate relative one another for adjustment of their relative angle.

Extending from the main body 122 are extensions 124 that preferably comprise ball-headed extensions. The extensions 124 are located within extension bores 126 defined by the main body 122. Set screws 128 are located in threaded bores 130 that intersect with the extension bores 126. When the set screws 128 are tightened, they exert a clamping action on the extensions 124 to lock the extensions 124 in place. When the set screws 128 are loosened, they allow the extensions 124 to move axially within the bores 130 for adjustment of the width of the device.

Further, attached to the extensions 124 are adjustable sockets 132 for lockably capturing the extensions 124. Preferably, the sockets 132 each comprise a body portion 134 having a mating surface 136 and a threaded collar 138 for threading onto a threaded outer surface of the body portion 134. When the collar 138 is tightened it exerts a clamping force on the extension 124, thereby maintaining the body portion 134 in a fixed relationship with the extension 124. When the collar 138 is loosened, the body portion 134 is movable with respect to the extension 124 such that the relative angle of the body portion 134 to the extension 124 can be manipulated.

The body portion 134 further comprises a cylindrical end 140 that extends into a clamp section 142. The clamp section 142 includes a fastener 144 that when tightened affixes the cylindrical end 140 to the clamp section 142 and when loosened allows axial movement of the cylindrical end 140 with respect to the clamp section 142 thereby, with the extensions 124, allowing for width adjustment of the device.

Attached to the clamp section 142 is another collar 148 and socket 146 arrangement. The collar 148 is attached to the clamp section 142 and the socket 146 defines a bore 150 through which a fastener 152 may be threaded into a pedicle screw 154. The socket 146 further defines a bore 156 through which the fastener 152 attaches to the pedicle screw 154. The bore 156 is sized such that when the fastener 152 is loosened in the pedicle screw 154 the angle of the pedicle screw 154 within the socket 146 may be adjusted within a range of adjustment. When the fastener 152 is tightened within the pedicle screw 154 the angle of the pedicle screw 154 within the socket 146 is fixed at the desired angle.

Further, the socket 146 defines openings 156 at opposite ends thereof through which a pedicle rod 158 may be inserted. The socket 146 further includes a pedicle rod mating surface 160 upon which pedicle rod 158 rests. When rotating the collar 148 to tighten it upon the socket 146, the collar 148 performs a clamping action upon the pedicle rod 158 to clamp the socket 146 into a fixed relationship with the pedicle rod 158. When the collar 148 is loosened, the socket 146 may be adjusted along the axis of the pedicle rod 158 and rotated around the circumference of the pedicle rod 158.

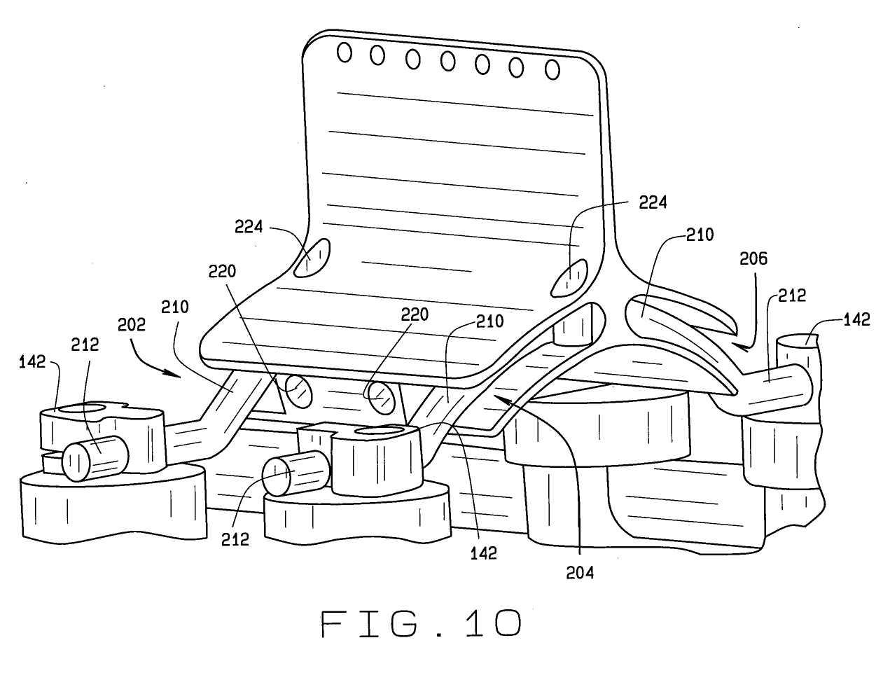

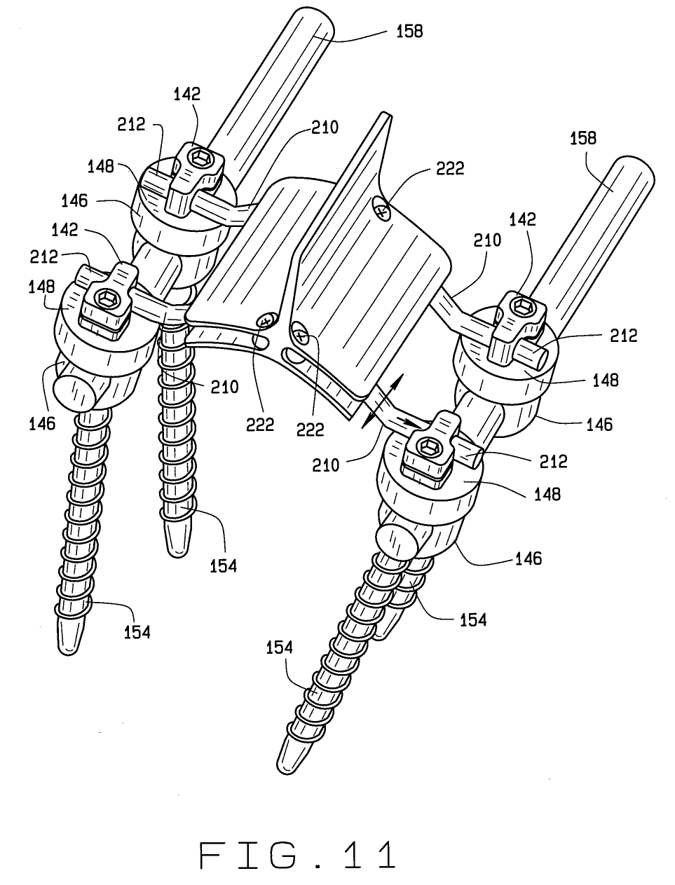

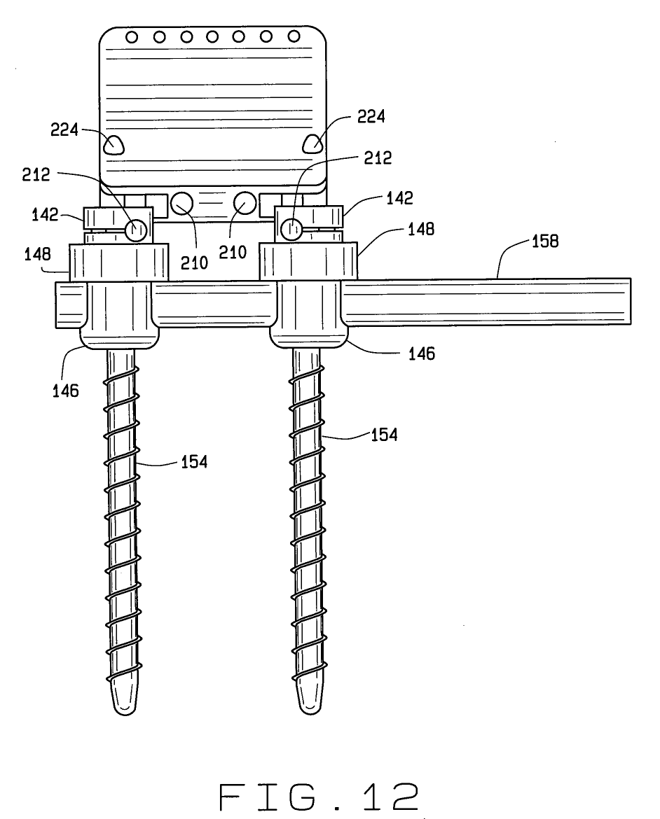

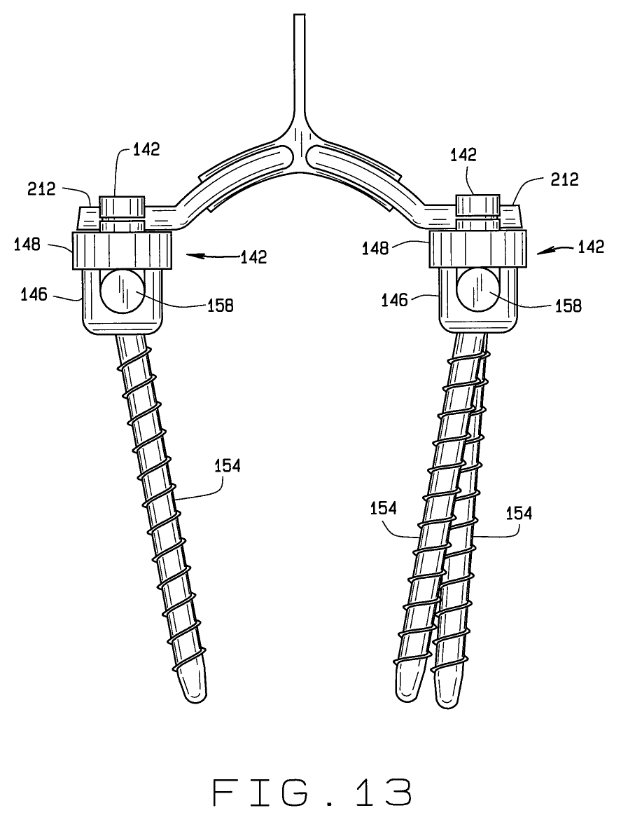

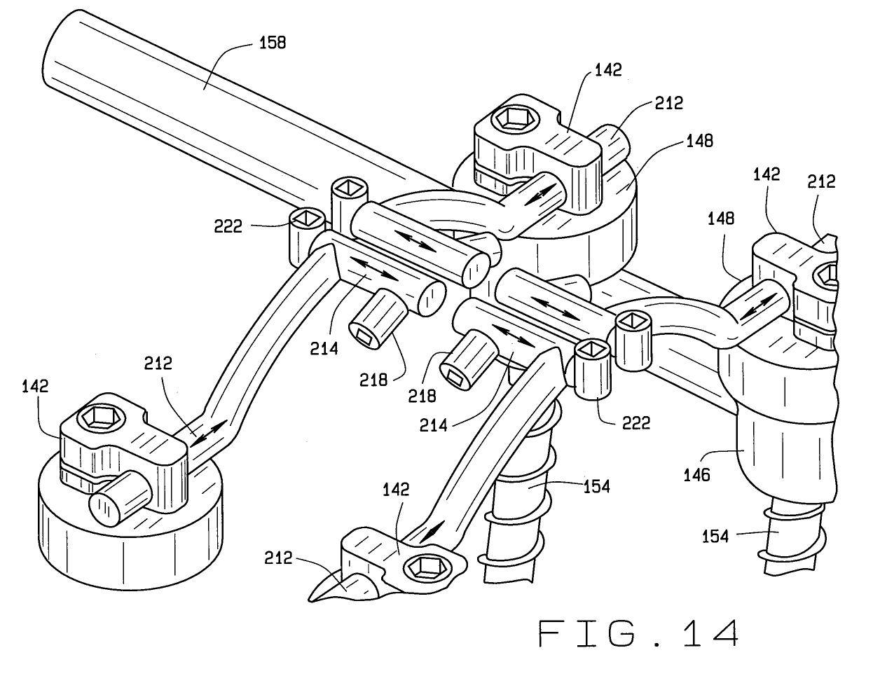

Referring to FIG. 10-14, in yet another embodiment of the present invention, the clamp section 142 and corresponding collar 148/socket 146/pedicle rod 158 arrangement may be utilized upon a main body 200 that incorporates cutout sections 202, 204, 206 and 208. Within the cutout sections 202-208 are located extensions 210 formed in a configuration to match a curvature of the main body 200 and then extend outwardly in the form of cylindrical ends 212 from the main body 200 in two opposed directions. The clamping sections 142 are then attached to the cylindrical ends 212 as with the cylindrical ends 140.

The extensions 210 are attached to the main body 200 by inserting main body ends 214 of the extensions 210 into corresponding bores 216 within the main body 200. Set screws 218 threaded into bores 220 when tightened clamp down the extensions 210 in a fixed position and when loosened allow the extensions to be moved axially with respect to the main body ends 214. Additionally, set screws 222 threaded into bores 224 act as bump stops to prevent inadvertent disassociation of the extensions 210 from the main body 200 due to slippage or loosening set screws 218.

By utilizing any of the present embodiments, a surgeon may perform a spinal fusion and place the present device upon the spine of a patient by adjusting the dimensions of the device to particularly fit the dimensions of the spine of the particular patient.

1. A device for covering and protecting the spinal cord of a patient after some or all of the spinous process or lamina has been removed comprising a main body adjustably attached to at least two rod attachment flanges such that each rod attachment flange may be adjusted closer and further from the main body, the rod attachment flanges each attached to a pedicle rod having at least four pedicle screws attached thereto, the pedicle attachment screws adapted for attachment to at least one vertebrae of the patient.

2. The device of claim 1 further comprising an extension portion extending outwardly therefrom to simulate the spinous process, the extension portion defining a plurality of bores therein for suture attachment.

3. The device of claim 1 wherein the rod attachment flanges are maintained within slots formed within the main body.

4. The device of claim 3 further comprising a plurality of set screws retained with threaded bores selectively affixing each rod attachment flange within one of the slots of the main body.

5. The device of claim 1 wherein each pedicle rod is adjustably attached to one of the rod attachment flanges.

6. The device of claim 5 wherein each pedicle rod is adjustably attached to one of the rod attachment flanges by a pedicle rod attachment portion with at least one set screw for selectively adjustably attaching the pedicle rod to one of the rod attachment flanges.

7. The device of claim 1 wherein one or more of the pedicle screws are adjustably attached to the pedicle rod along or about the axis of the pedicle rod.

8. The device of claim 7 wherein one or more of the pedicle screws are adjustably attached to the pedicle rod along and about the axis of the pedicle rod by pedicle attachment flanges that receive a pedicle attachment flange set screw for adjustably clamping the pedicle attachment flange to the pedicle rod.

9. The device of claim 8 wherein at least one of the pedicle rod attachment flanges comprises an extension portion and a body portion wherein the relative angle of the body portion to the extension portion may be selectively adjusted.

10. The device of claim 9 wherein one of the extension portion and body portion comprises a ball portion and the other of the extension portion and the body portion comprises a locking collar portion for selectively locking and unlocking the ball portion to select the relative angle of the body portion to the extension portion.

11. The device of claim 9 wherein the body portion is received within a clamp of a socket portion for adjustably attaching the socket portion along an axis of the body portion.

12. The device of claim 11 wherein the socket portion comprises a collar and socket for receiving the pedicle rod, the collar threadingly received upon the socket for selectively clamping the pedicle rod to adjustably clamp the socket along and about the pedicle rod.

13. The device of claim 12 wherein the pedicle screw is adjustably attached to the socket by a fastener such that the pedicle screw is adjustable in angle with respect to the socket.

14. The device of claim 1 wherein the pedicle rod and main body are adapted for relative adjustment of the angle of adjustment of the pedicle screws with respect to the main body.

15. The device of claim 1 wherein the main body comprises at least two hinged members attached to one another, the hinged members adapted for relative rotation with respect to one another and lockable with respect to one another.

16. The device of claim 15 further comprising an extension portion rotatable and lockable to the two hinged members and lockable thereto, the extension portion defining a plurality of bores therein for suture attachment.

17. The device of claim 1 wherein the main body defines cutout sections.

18. The device of claim 17 further comprising extensions attached to the main body and received within the cutout sections, the extensions having pedicle screws attached thereto.

19. The device of claim 18 wherein the extensions define main body ends received within bores defined the main body, the bores formed axial to the main body and allowing relative adjustment of the pedicle screws nearer and further one another by selectively locking the main body ends within the bores.

20. The device of claim 19 further comprising bump stops preventing disassociation of the extensions from the bores.
